(19) United States
(12) Patent Application Publication
(10) Pub. No.: US 2024/0091143 A1
(43) Pub. Date: Mar. 21, 2024

(54) SEMI-SOLID CHEWABLE COMPOSITIONS AND METHODS OF MAKING AND USING THEREOF

(71) Applicant: SEATTLE GUMMY COMPANY, Seattle, WA (US)

(72) Inventors: Feng WAN, Issaquah, WA (US); William Brenden CARLSON, Shoreline, WA (US)

(21) Appl. No.: 18/014,757

(22) PCT Filed: May 3, 2021
(86) PCT No.: PCT/US21/30390

§ 371 Date: Jan. 6, 2023

Related U.S. Application Data
(60) Provisional application No. 63/019,379, filed on May 3, 2020.

(51) Int. Cl. A61K 9/68; A61K 31/198; A61K 31/375; A61K 31/4188; A61K 31/4425; A61K 31/505; A61K 31/59; A61K 33/16; A61K 36/23; A61K 36/258; A61K 36/28; A61K 36/288; A61K 36/315; A61K 36/355; A61K 36/534; A61K 36/539; A61K 36/54; A61K 36/634; A61K 36/66; A61K 36/758; A61K 36/899; A61K 47/26

(52) U.S. Cl. CPC A61K 9/0058; A61K 31/198; A61K 31/375; A61K 31/4188; A61K 31/4425; A61K 31/505; A61K 31/59; A61K 33/16; A61K 36/23; A61K 36/258; A61K 36/28; A61K 36/288; A61K 36/315; A61K 36/355; A61K 36/534; A61K 36/539; A61K 36/54; A61K 36/634; A61K 36/66; A61K 36/758; A61K 36/899; A61K 47/26

(57) ABSTRACT

A semi-solid chewable composition, comprising an herbal composition and a semi-chewable base. The herbal composition comprises anti-inflammatory, anti-microbial, analgesic, antiseptic, or antipyrelic herbal composition.

### CROSS REFERENCE TO RELATED APPLICATION

The application claims the priority benefit of U.S. Provisional Patent Application No. 63019379, filed on May 3, 2020, which is hereby incorporated by reference in its entirety.

### TECHNICAL FIELD

This application relates to semi-solid edible or chewable compositions with one or more bioactive incorporated therein.

### BACKGROUND

Unless otherwise indicated herein, the materials described in this section are not prior art to the claims in this application and are not admitted being prior art by inclusion in this section.

The health and hygiene of the teeth, the mouth, and mucosal tracts of the oral cavity is important to a person's overall health. The common type of oral disease includes dental cavities, caries, bad breath and gum diseases, including canker sore, gingivitis, and periodontitis. Canker sores and gingivitis are the most commonly occurring oral problems. Recurrent aphthous stomatitis (RAS) (aphthae; canker sores) is one of the most common, painful oral ailments.

Periodontal diseases (gingivitis and periodontitis) are largely caused by specific gram-negative anaerobic bacterial infections, leading to the initial destruction of the soft connective tissue and, subsequently, to the disruption of the underlying alveolar bone and ligament supporting the teeth. The bacterial species Porphyromonas gingivalis has been implicated as a major etiologic agent in the development and progression of periodontitis. Other species also contributing to gingival inflammation are Treponema denticola, Prevotella denticola and Fusobacterium nucleatum. Based on the World Health Organization surveys, most children have signs of gingivitis, and among adults the initial stages of periodontal disease are highly prevalent.

Dental caries (also known as tooth decay) is a disease wherein bacterial processes damage hard tooth structure. Streptococcus mutans is one of a few specialized organisms equipped with receptors that help for better adhesion to the surface of teeth, thus being an early coloniser of the dental surface and the most significant contributor to caries. The growth and metabolism of this pioneer species creates an acidic environment in the mouth which causes the highly mineralized tooth enamel to be vulnerable to decay.

Dental plaque is responsible for many of the diseases common to the oral cavity including dental caries, periodontitis, gingivitis, and the less common peri-implantitis (similar to periodontitis, but with dental implants).

Dental calculus is an ash gray, yellowish or dark brown calcified substance depositing on dental crowns, exposed dental root surfaces, or surfaces of restorative dental materials. Plaque (dental plaque) adheres to surfaces of human teeth. In the plaque, a reaction occurs by which inorganic salts become more adhesive, and calcification starts from a layer which touches a tooth surface. The calcification advances as the plaque becomes older and thicker, and new plaques adhere to the surface of the calcified plaques and causes calcification. Dental calculus is formed by repetition of this process.

In addition to the above, one further oral disorder is believed to affect a large proportion of the population: halitosis. Also referred to as bad breath, halitosis is caused by a number of volatile compounds derived from the bacterial degradation of sulphur-containing amino acids. These bacteria (mostly Fusobacterium nucleatum, Porphyromonas gingivalis, Porphyromonas intermedia, and Treponema denticola) are located in stagnant areas in the oral cavity, such as the dorsal surface of tongue, periodontal pockets, and interproximal areas. This affection has a significant impact—is estimated to be the third-most-frequent reason for seeking dental aid, following tooth decay and periodontal disease.

Oral bacteria form a biofilm (dental plaque) on all hard and soft oral tissues, which is considered to be the principal etiologic agent in the pathological conditions of the mouth. The accumulation of bacteria within the biofilm, facilitated by poor oral health maintenance, predisposes to allogenic shifts in the microbial community, leading to the onset of periodontal inflammation and caries formation, as well as contributing to halitosis.

Mechanical means are the generally employed in maintaining oral hygiene and controlling plaque. These techniques are time consuming and require skill to be performed well for effective relief. Antimicrobial agents such as chlorhexidine, metronidazole etc are being used extensively as an adjunct to mechanical cleaning. However, these drugs have unpleasant side-effects. There is a need for a safe, efficient and effective natural or herbal alternative for managing oral health and hygiene.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides semi-solid chewable composition. In one embodiment, the semi-solid chewable composition manages oral health. The oral health semi-solid chewable composition comprises a chewable base composition and an active composition.

The chewable base composition may comprise polymers, waxes, elastomers, humectants, plasticizers, sweeteners, flavors, fillers, colorants. In one embodiment, the polymer may be natural or synthetic sourced. In one embodiment, the wax may be natural or synthetic sourced.

In one embodiment, the chewable base may be dissolvable through chewing. In one embodiment, the semi-solid chewable composition comprises a gummy composition. The composition could be easily chewed and quickly dissolve in oral cavity from about less than 1 to about 2 minutes. The composition could be chewed and slowly dissolve in oral cavity from about at least 2 minutes to about 5 minutes. In one embodiment, the composition dissolves in oral cavity through chewing within 1 minute. In one embodiment, the composition dissolves in oral cavity through chewing in less than 2 minutes. In one embodiment, the composition dissolves in oral cavity through chewing in more than 3 minutes.

In one embodiment, the chewable base may be non-dissolvable upon chewing. In one embodiment, the semi-solid chewable composition comprises a gum composition.

In one embodiment, the chewable composition may comprise a coating.

The semi-solid chewable composition may include active ingredients that promote oral health. Example actives include vitamins, minerals, amino acids, herbal actives, fluoride salts, polishing agents, teeth whitening agents, anti-gingivitis agents, anti-caries agents, anti-calculus agents, analgesics, anti-inflammatory, antiseptics, anti-microbial agents, anti-ulcer agents, prebiotics, probiotics, and pH buffering agents.

Example vitamins may include biotin, vitamin D, vitamin K, Vitamin C, Vitamin A, Vitamin Bs, their derivatives, or a combination thereof. In some embodiment, the disclosed composition comprises from about 0.01% to about 0.5%, from about 0.2% to about 1%, from about 0.7% to about 3%, about 0.2%, about 1% of the vitamin composition.

Example minerals may include calcium salts, potassium, phosphorus, fluoride salts, zinc salts, their derivatives, or a combination thereof. In some embodiment, the disclosed composition comprises from about 0.5% to about 50%, from about 2% to about 40%, from about 3% to about 10%, about 1%, about 5%, about 3%, about 5%, about 10%, about 20%, about 30%, about 35% of the mineral composition.

Example amino acids may include arginine, histidine, isoleucine, leucine, lysine, methionine, phenylalanine, threonine, valine, their derivatives, or a combination thereof. In some embodiment, the disclosed composition comprises from about 0.01% to about 10%, from about 1% to about 5%, from about 1.5% to about 8%, about 1.5%, about 3%, about 5%, about 8% of the amino acid composition. In one embodiment, the disclosed composition comprises from about 1% to about 10% arginine. In one embodiment, the disclosed composition comprises arginine, lysine, or a combination thereof. In one embodiment, the disclosed composition comprises arginine bicarbonate.

Example herbal actives may include clove, clove oil, coconut oil, pomegranate, green tea, Salvadora persica (meswak), aloe vera, acacia arabica, Melaleuca alternifolia (tea tree), Azadirahta indica (neem), locorice, turmeric, ginger, ginseng, Azadirachta Indica, Ocimum sanctum, Murraya koenigii L., Acacia nilotica, Eucalyptus camaldulensis, Hibiscus sabdariffa, Mangifera indica, Psidium guajava, Rosa indica, Aloe barbadensis Miller, sanguinarine, propolis, Bloodroot, Caraway, Chamomile, Echinacea, Myrrh, Peppermint, Rosemary, Sage, Thyme, their derivatives, or a combination thereof.

In some embodiment, the disclosed composition comprises from about 0.01% to about 15%, from about 0.5% to about 10%, from about 1% to about 7%, about 2%, about 5%, about 3%, about 5%, about 10% of the herbal composition.

The herbal actives may include Traditional Chinese Medicine (TCM) formulations or herbs for promoting oral health, reducing swelling, relive pain, or a combination hereof. The TCM herbs or formulations useful in currently application may be anti-bacterial, anti-inflammatory, anti-viral, analgesics, antiseptics, ant-ulcer, their derivatives, or a combination thereof.

Polishing agents may include aluminum oxide, aluminum hydroxide, calcium carbonate, dibasic calcium phosphate dihydrate, mica, sodium bicarbonate, pumice, silicon carbide, aluminum silicate, silicon dioxide, carbide compounds, garnet, feldspar, zirconium silicate, zirconium oxide, boron, emery, silica, perlite, their derivatives, or a combination thereof. In some embodiment, the disclosed composition comprises from about 0.01% to about 5%, from about 0.2% to about 1%, from about 0.7% to about 3%, about 0.2%, about 1% of the polishing agent.

Example whitening agents may include silica, pyrophosphates, hydrogen peroxide, carbamide peroxide, their derivatives, or a combination thereof. In some embodiment, the disclosed composition comprises from about 0.01% to about 30%, from about 1% to about 5%, from about 1.5% to about 3%, about 1%, about 2% of the whitening agent.

Example anti-gingivitis agents may include chlorhexidine, essential oils, triclosan, Gantrez copolymer, stannous fluoride, zinc citrate, sodium hypochlorite, or a combination thereof. In some embodiment, the disclosed composition comprises from about 0.01% to about 1%, from about 0.1% to about 0.5%, from about 0.05% to about 1.5%, about 0.1%, about 0.2% of the anti-gingivitis agent.

Example anti-caries agents may include sodium fluoride, stannous fluoride, sodium monofluorophosphate, their derivative or a combination thereof. In some embodiment, the disclosed composition comprises from about 0.001% to about 0.5%, from about 0.05% to about 0.2%, from about 0.1% to about 0.15%, about 0.1%, about 0.15% of the anti-caries agent.

Example anti-calculus agents may include pyrophosphate, polyvinyl methyl/ethyl/maleic acid copolymer, pyrophosphate, their derivative or a combination thereof.

Example analgesics may include potassium nitrate, stannous fluoride, nonsteroidal anti-inflammatory drugs (NAIDS), lavender essential oil, rosemary essential oil, peppermint essential oil, eucalyptus essential oil, cloves, white willow, Boswellia, capsaicin, ginger, turmeric, feverfew, hydroxy-alpha-sanhool, Szechuan pepper, their derivatives, lidocaine, or a combination thereof. In some embodiment, the disclosed composition comprises from about 0.001% to about 10%, from about 0.05% to about 5%, from about 0.1% to about 3%, about 0.1%, about 0.15%, about 0.2%, about 1% of the analgesic.

Example antiseptics may include charcoal, euthymol, triclosan, essential oils, sage, sea salts, their derivatives, or a combination thereof. In some embodiment, the disclosed composition comprises from about 0.01% to about 5%, from about 0.1% to about 1%, from about 0.2% to about 3%, about 0.3%, about 2% of the antiseptics.

Example anti-ulcer agents may include dexlansoprazole, esomeprazole, lansoprazole, omeprazole, pantoprazole, rabeprazole, anti-ulcer herbs such as paras papal, alstonia scholaris R. Br., Asparagus racemosus Wind, Azadirachta indica, Kachnar or Kaniar (Bauhinia variegate Linn.), Palaas IButea frondose Roxb.), Papitta (Carica papaya L.), quercetin, flavonoid, Annona squamoza, Mentha microphylla, Oreganum marjoranum, O. syriacum, Solanum nigrum, Chicorium intybus, Matricaria recutita (Geraman chamomile), Brassica oleracea (white cabbage and cauliflower), Lagenarea sicerarea, Portolaca oleracea, mastic, their derivatives, or a combination thereof.

Example pH buffering agents may include sodium sales such as carbonate, bicarbonate, borate, or phosphate, citric acid, acetic acid, tromethamine, histidine, gluconic, lactic, tartaric acid, aspartic acid, glutamic acid, malic acid, or a combination thereof.

The oral health composition may have a pH from about 2.1 to about 7, from about 2.5 to about 3.2, from about 2.9 to about 3.5, from about 3.2 to about 4.1, from about 3.7 to about 5, from about 4.2 to about 5.6, from about 5 to about 6, from about 6 to about 7.

The oral health composition may have the function of anti-caries, reduce inflammation caused by gum disease, reducing gum bleeding, relieve toothache and gum pain, reduce bad breath, clean and refresh the oral cavity, or a combination thereof.

In another aspect, the application provides methods of making the disclosed compositions.

In a third aspect, the application provides methods of using the disclosed compositions to promote oral health and general wellbeing. In one embodiment, the application provides compositions for prevention and/or treatment of a disorder caused by oral pathogenesis in an oral cavity. The disorder of the oral cavity is a dental plaque-related disorder. In one embodiment, the dental plaque-related disorder is gingivitis, periodontitis, caries, or sensitive teeth. In one embodiment, the disorder of the oral cavity is halitosis. In one embodiment, the disorder of the oral cavity is candidiasis.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, and processes related to semi-solid chewable composition.

Chewing action is regarded as a helpful way to cure halitosis (bad breath) and can help to maintain tooth health. The breath and tooth benefits are through the process of salvation and physical action on the teeth. The physical action of chewing can remove particulates such as trapped food from the teeth. Salvation helps to maintain pH and washes the mouth to remove bacteria.

Teeth are composed of a mineral called hydroxyapatite [Ca₅(PO₄)₃]OH. The mineral can be attacked by acids leading to dental caries. Substituting fluoride for hydroxide to make fluorapatite, [Ca₅(PO₄)₃]F results in a mineral that is much less susceptible to dental caries. Sources for the fluoride ion can be sodium fluoride, potassium fluoride, calcium fluoride and stannous (tin II) fluoride.

The whitening of teeth can be accomplished by the addition of oxidizing or reducing agents. Some whitening agents include hydrogen peroxide, hydrogen peroxide urea complex, sodium perborate, sodium percarbonate, carbamide peroxide, azobisisobutyronitrile, and sodium hydrosulfite.

Gingivitis is a disease of the gums and is associated with plaque build upon the gumline. There may also be lesions in the mouth that resemble canker sores with gingivitis. The oral health chewable composition may include the following compounds to prevent gingivitis, tartar, and plaque build hexetidine, delmopinol, stannous fluoride, caffeic and p-coumaric acids, cetylpyridinium chloride, and chlorhexidine.

In one embodiment, the oral care semi-solid chewable composition comprises a semi-solid chewable base and an active composition. In one embodiment, the composition can include one or more of the following: a gelling agent, a humectant, a flavoring agent, a bleaching agent/peroxide stabilizer, agent to diminish tooth sensitivity, a chelating agent, a sweetener, or a combination thereof.

In one embodiment, the composition may include one or more of polishing agents, thickening agents, surfactants, humectants, solvents, sweeteners, tooth hardeners, anti-tartar agents, anti-calculus agents, flavoring agents, antibacterial agents, or a combination thereof.

Semi-Solid Chewable Base

Dissolvable Chewable Base

In some embodiment, water-soluble oral health semi-solid chewable compositions are provided. The composition may dissolve, through regular chewing, from about 20 seconds to about 5 minutes, such as 20 seconds, 30 seconds, 40 seconds, 50 seconds, 1 minute, 2 minutes, 3 minutes, 4 minutes, or 5 minutes. In one embodiment, the composition may slowly dissolve when chewed. The main physical properties of the water-soluble chewable is derived from the polymeric components wherein the polymeric components are water soluble. The oral health chewable may be based on mixtures of starch and any one of the gums such as gum arabic, locust bean gum, xanthan gum, guar gum, gelatin, pectin and the like, which emulsifies the substance and binds them in a hydrocolloidal matrix. The starch and gum hydrocolloidal matrix reduce the rate in which the oral health chewable dissolves and moderates the amount of active substances delivered at a time.

Gelling Agents

In one embodiment, the semi-solid chewable oral health composition comprises a gummy formulation. In one embodiment, the gummy composition comprises a gelling agent. In one embodiment, the gelling agent is a high molecule weight polymer. In one embodiment, the gelling agent comprises pectin, gelatin, agar, carrageenan, starch, gum Arabic, konjac, or a combination thereof.

In one embodiment, the gelling composition comprises starch. In one embodiment, the starch comprises amylose starch. For gummy formulations, the starch is often “modified” with modification techniques by contacting starch with acid, sodium or potassium hydroxide, or oxidizing the starch. These treatments help the starch to dissolve in water and gel to an appropriate level.

Agar is derived from the polysaccharide agarose and contains two components: the linear polysaccharide agarose, and a heterogeneous mixture of smaller molecules called agaropectin. Agar gels tend to weep or extrude water over time when used by itself as a gelling agent. In one embodiment, agar is combined with locust bean gum as a gelling agent. Locust bean gum helps to prevent weeping of agar gels. Locust bean gum is a galactomannan polysaccharide vegetable gum extracted from the seeds of the carob tree. The two polysaccharides from agar and locust bean gum synergistically interact with each other to form a strong gel that does not weep.

Carrageenans or carrageenins are a family of linear sulfated polysaccharides that are extracted from red edible seaweeds. The linear saccharide chains have a tendency to curl to form helical structures. Kappa-carrageenan has one sulphate group per disaccharide and forms strong, rigid gels in the presence of potassium ions. Similar to agar, locust bean gum is often used with kappa-carrageenan to prevent water from being expelled from the bulk of the gel (weeping). Gels formed from kappa-carrageenan and potassium ions are thermally reversible, meaning they melt when heated and solidify when cooled.

Alginic acid is a linear copolymer with homopolymeric blocks of (1-4)-linked β-D-mannuronate (M) (acid form of mannose) and its C-5 epimer α-L-guluronate (G) (acid form of gulose) residues, respectively, covalently linked together in different sequences or blocks. The monomers can appear in homopolymeric blocks of consecutive G-residues (G-blocks), consecutive M-residues (M-blocks) or alternating M and G-residues (MG-blocks). Alginate forms strong hydrogels when crosslinked with calcium ions.

Bonding Agents: Simple Carbohydrates

The carbohydrates may act as bonding agents to bond gummy matrix together through interaction with the gelling agent. The carbohydrates may also act as a humectant.

Example carbohydrates include sucrose (table sugar), fructose, glucose, psicose, sorbose, and tagatose, trehalose (mycose, tremalose), palatinose, their derivatives or a combination thereof. In one embodiment, the bonding agents includes palatinose, which has lower instances of tooth decay.

In one embodiment, the composition comprises a low index sugar having a glycemic index of not more than 20. In one embodiment, the semi-solid chewable composition is substantially free of sugar having a glycemic index of more than 35.

Through extensive experimentation, processes are developed to used psicose, sorbose, or tagatose in the semi-solid gummy formulation allowing these sugars behave like sucrose and fructose but without the caloric significance of sucrose.

In one embodiment, the low glycemic index sugar comprises psicose, sorbose, tagatose, trehalose, isomaltulose, raffinose or a combination thereof. In one embodiment, the carbohydrate component of the semi-solid chewable composition consists essentially of psicose, isomaltulose, and a third low GI sugar selected from a group consisting of trehalose, sorbose, tagatose, or a combination thereof. In one embodiment, the composition further comprises N-acetyl glucosamine.

In one embodiment, the semi-solid chewable composition comprises psicose, trehalose and palatinose. In one embodiment, palatinose comprise more than 50% of the carbohydrate component comprising psicose, trehalose and palatinose. In one embodiment, the semi-solid chewable composition has a glycemic index of not more than 8, 10, 15 or 20.

In one embodiment, the semi-solid chewable composition is substantially free of sucrose, fructose and glucose. In one embodiment, the semi-solid chewable composition is substantially free of sugar alcohol. In one embodiment, the semi-solid chewable composition is substantially free of sugar substitutes such as sucralose, stevia, monk fruit extract, Splenda, etc.

Bonding Agents: Sugar Free Carbohydrates

Bonding agents may include sugar alcohols including without limitation sorbitol, mannitol, erythritol, xylitol, isomalt, maltitol, lactitol, isomalt, and hydrogenated starch hydrolysates. Advantages of sugar alcohols include non-cariogenic, preventing tooth decay, flavors masking, or as an excellent humectant. In one embodiment, mannitol is used to mask bitterness. In one embodiment, xylitol is used to prevent tooth decay. In one embodiment, erythritol is used as a humectant in the composition. In one embodiment, the disclosed composition is substantially free of sugars including without limitation sucrose, glucose, or fructose.

Non-Dissolvable Chewable Base

In one embodiment, the semi-solid chewable oral health composition comprises a gum formulation.

In one embodiment, the gum base comprises resin, wax, and elastomer. In one embodiment, resin is the main chewable portion, wax softens the gum, and elastomers add flexibility. Additional additives include plasticizers, which act to soften the resin, surfactants, which help to stabilize the gum base mixture and sweeteners.

In one embodiment, the base resin and waxes comprise hydrophobic materials, leading to the composition to not dissolve and retain physical properties when chewed. Some example resin materials suitable for the non-dissolvable gum base are: Apocynaceae, Chicle, Chilte, Chiquibul, Crown Gum, Euphorbiaceae, Gutta hang kang, Jelutong, Leche 7aspi (sorva), Leche de vaca, Massaranduba balata, Massaranduba chocolate, Moraceae, Niger gutta, Nispero, Paraffin, Pendare, Perillo, Polyethylene, Pine rosin, Rosidinha, Sapotaceae, Tunu (tuno), and Venezuelan chicle. To give the sensation of loss, a water-soluble polymer can also be mixed with the hydrophobic resin. Example water-soluble polymers include gelatin, pectin, carrageenan, guar gum, xanthan gum, gum Arabic, locust bean gum, polyacrylic acid and associated salts, polyethylene glycol, polyethylene oxide, polylactic acid, hydroxyethyl cellulose, carboxymethyl cellulose, or a combination thereof.

Waxes blend with the resin components to adjust physical properties of the resin such as softening the resin to make it more palatable and easily chewable. In one embodiment, the waxes are hydrophobic. Example waxes include Bees Wax, Camauba Wax, Paraffin Wax, Polyethylene Wax, and Petroleum Wax.

The elastomer component to the gum base imparts the chewy and softness properties to the oral health chewable. Example elastomers include Butadiene-styrene rubber, Isobutylene-isoprene copolymer (butyl rubber), Natural rubber and Polyisobutylene Polyvinyl acetate.

In one embodiment, the semi-solid chewable composition may include crosslinked acrylic acid polymers as gelling agent. Example polymers include the “carbomer” family of polymers, e.g., carboxypolyalkylenes that may be obtained commercially under the Carbopol® trademark. Examples of hydrophilic polymers include polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate, and gelatin.

In order to prepare a uniform gel, dispersing agents such as alcohol or glycerin may be added, or the polymer can be dispersed by trituration, mechanical mixing or stirring, or combinations thereof. These polymers can also act as emulsion stabilizers such as carbomers. The oral care composition includes carbomers. The gelling agent content can be in the range of from about of 1 w % to about 15 w %, from about 2 w % to about 4 w %.

Humectants

An oral or pharmaceutically acceptable humectant can include one or a mixture of humectants, such as, glycerin, sorbitol, polyethylene glycol, a mixture of glycerin and sorbitol, a mixture of propylene glycol and glycerin, or a combination thereof. The humectant content can be in the range (weight %) of from about 1% to about 30%, about 2% to about 25%, from about 2% to 6%, or any % number in between. Example humectants are glycerol (glycerin, 1,2,3-propantriol), propylene glycol (1,2-propandiol), aloe vera gel, glyceryl triacetate, and a-hydroxyacids (lactic acid).

Sweeteners

Example sweetener may be non-cariogenic therefore the composition is substantially free of cariogenic sugars such as sucrose, fructose, and glucose. Example non-cariogenic sweeteners may include psicose, sorbose, tagatose, trehalose, isomaltulose, or a combination thereof. Sugar alcohol examples include erythritol, sorbitol, mannitol, maltitol, isomalt, and xylitol. Some sugar substitutes are saccharin, aspartame, sucralose and acesulfame K. In some embodiments, the sweetener not only provides sweetness but also decreases the populations of bacteria in the mouth that lead to oral health problems. In one embodiment, the sweetener is selected from one or more of saccharin, aspartame, cyclamate, sucralose, Stevia, mannitol, sorbitol, xylitol and similar glycols.

Plasticizers

Plasticizers act in oral health chewable to decrease brittleness and increase chewiness. Example plasticizers are lecithin, hydrogenated vegetable oils, glycerol mono, di and tri acetate ester, lanolin, methyl ester of the fatty acids, pentaerythritol mono, di, and tri acetate ester, rice bran wax, stearic acid, sodium, potassium stearates, their derivatives or combination thereof.

Flavoring Agents

An flavoring agent may be one or a mixture of flavoring agents, such as: bubble gum flavor, cherry flavor, grape flavor, anise oil, cassia oil, vanilla extract, vanilla creme, orange flavor, anethole, licorice, spearmint oil, phenylacetaldehyde diisobutyl acetal, essential oils such as spearmint, orange, mint, tea tree essential oil. Some flavoring agents can also act as sweeteners include, for example, neohespiridin dehydrochalcone, xylitol, sucralose, and mixtures thereof. The flavoring agent content the oral care composition embodiments and the oral care composition embodiments can be in the range (weight %) of from about of 0.05% to about 3%, from about 0.5% to about 1%, from about 0.2% to about 1.5%, or any number in between.

Flavors and colors are for sensory appeal. Flavor may be in liquid, powder or micro-encapsulated forms. Liquid flavor incorporations may be either water-soluble, oil-soluble, or water-dispersible emulsions. For hydrophobic gum composition, the oil-soluble flavors remain in the composition longer, resulting in longer lasting flavor sensations.

In one embodiment, the flavoring agent may a phenolic flavoring agent selected from eucalyptol, thymol, methyl salicylate, menthol, chlorothymol, phenol, wintergreen oil, spearmint oil, peppermint oil and similar essential oils, and halogenated and other derivatives thereof.

Surfactants

An oral or pharmaceutically acceptable surfactant may include one or a mixture of surfactants, such as nonionic, anionic and amphoteric surfactants. In one embodiment, the surfactant may include sulfate, sulfonate and phosphate ester surfactants (e.g., alkyl sulfonates having 10 to 18 carbon atoms and sulfates of monoglycerides of fatty acids having 10 to 18 carbon atoms) as well as salts and derivatives thereof. In one embodiment, the surfactant may include sodium lauryl sulfate (SLS) or sodium lauryl ether sulfate (SLES). In on embodiment, the surfactant may anionic taurate surfactants such as sodium methyl cocoyl taurate and sodium methyl oleoyl taurate, PEG caster oils, and PEG hydrogenated caster oils. Example PEG hydrogenated caster oils include PEG-60 Hydrogenated Castor Oil.

In one embodiment, the oral care composition embodiments include sodium lauryl sulfate (SLS), sodium methyl cocoyl taurate, or a mixture thereof. In one embodiment, the composition comprises oil based surfactant such as PEG-60 Hydrogenated Castor Oil.

The surfactant content can be in the range (weight %) of from about of 0.1% to about 10%, from 1% to about 2%, from about 2% to about 5%, or any number in between.

In one embodiment, the surfactant may be one or more of sodium lauryl sulfate, sodium N-coco, N-methyl taurate, sodium N-lauroyl sarcosine, or a compatible dental detergent.

Binders

An oral or pharmaceutically acceptable binder can include one or a mixture of binders. Example binder includes water soluble binders (such as polyvinyl alcohols, polyvinyl pyrrolidone (PVP), polyvinyl pyrrolidone derivates and copolymers, or combination thereof), saccharides (such as D-glucose, D-fructose, sorbitol, or combination thereof), polysaccharides (such as starch, cellulose, pectin, their derivatives, or combination thereof), proteins such as gelatin, naturally occurring substances (such as alginic acid, carrageen gum, lucost bean gum, guar gum, xanthan gum, tragacanth gum, arabic gum, karaya gum, or a combination thereof).

In one embodiment, the oral care composition comprises polyvinyl pyrrolidone (PVP), cellulose gum, xanthan gum, or a combination thereof as a binder and/or thickener. The binder/thickener may be present in the oral care composition in concentrations (weight %) from about 0.1% to about 10%, from about 5% to about 8%, from about 2% to about 4%, or any number in between.

Thickening Agents

An oral or pharmaceutically acceptable thickener can include one or a mixture of organic and inorganic thickeners or viscosity modifiers. Organic thickeners may include natural and synthetic gums and colloids. Examples include carrageenan (Irish moss), xanthan gum and sodium carboxymethyl cellulose, starch, polyvinylpyrrolidone, hydroxyethylpropyl cellulose, hydroxybutyl methyl cellulose, hydroxypropylmethyl cellulose, hydroxyethyl cellulose, or a combination thereof. Inorganic thickeners may include Laponite D, amorphous silica compounds such as colloidal silica compounds available under tradenames such as Cab-o-sil fumed silica manufactured by Cabot Corporation and distributed by Lenape Chemical, Bound Brook, N.J., Zeodent 165 from J. M. Huber Chemicals Division, Havre de Grace, Md. 21078 and Sylox 15 from Grace Davison, Baltimore, Md. 21203.

In one embodiment, the thickener may include one or more of a natural or synthetic gum, carrageenan, hydroxymethyl cellulose, a siliceous thickener, fumed silica, or a combination thereof.

Coatings

A coating can be applied to the oral health chewable product. In one embodiment, the coating may include sugars or sugar alcohols. Example sugars may include sanding sugar (sucrose), non-carogenic sugars such as psicose, sorbose, tagatose, trehalose, and isomaltulose, or sugar alcohols such as erythritol, sorbitol, mannitol, maltitol, isomalt, and xylitol. Resistant starches may also be used for coating.

Alternatively, the oral health chewable product can be wrapped in a film such as a wax paper or a rice paper.

Preservatives

An oral or pharmaceutically acceptable preservative can include, for example, benzoic acid, sodium benzoate, methylparaben, propylparaben, sorbic acid, potassium sorbate, or a combination thereof. These preservative agents may be present at levels (weight %) ranging from about 0.01% to about 2%. In one embodiment, the preservative comprises sodium benzoate.

Active Composition

Vitamins

In one embodiment, the oral health chewable comprises of vitamins A, B1, B2, B3, B6, B12, D and K to promote oral health and strengthen teeth. Vitamin deficiency can lead to poor oral health. Vitamin A deficiency presents itself as pits, grooves, or missing areas of enamel caused by a defective enamel matrix formation during tooth development, thus giving the tooth a translucent appearance. Vitamin B1 (thiamine) deficiency can result in cracked lips or red, swollen patches in the corners of the mouth (angular cheilitis. Vitamin B2 (riboflavin) and vitamin B3 deficiency can cause inflammation of the tongue, angular cheilitis, and ulcerative gingivitis. Oral manifestations that can be detected as a result of vitamin B6 deficiency is periodontal disease, a burning sensation, and sore tongue and vitamin B12 deficiency can show signs of angular cheilitis, halitosis, periodontal disease, hemorrhagic gingivitis, and painful ulcers in the mouth. Vitamin D is nutrient that aids in the absorption of calcium and phosphorous from the foods consumed. Also, vitamin D increases the production of antimicrobial proteins that fights bacteria causing decay. Therefore, one sign of a vitamin D deficiency is an increase in dental caries. A lack of vitamin D results in abnormal alveolar bone patterns and hypo-mineralization that compromises tooth integrity. Vitamin K helps to strengthen teeth.

For the hydrophobic gum composition, some vitamins are water soluble and are easily extracted out of the gum while the composition is chewed. However, some vitamins are oil soluble and will be attracted to the hydrophobic gum base. Because of the attraction of hydrophobic vitamins to the hydrophobic chewable base the release of the oil soluble vitamins will be slower. Surfactants may be used to complex the oil soluble vitamins and allow them to enter the saliva more quickly. Example surfactants can be lecithin, cholesterol, lanolin, tea saponin, protein, saponins, sugars and alkyl polyglycosides, or a combination thereof.

Vitamins in the oral health composition may include vitamin A, Vitamin B1 (thiamine), Vitamin B2 (riboflavin), Vitamin B3 (niacin, inositol hexanicotinate or niacinamide), Vitamin B5 (pantothenic acid or pantothenic acid salt), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins, commonly cyanocobalamin in vitamin supplements), vitamin C, vitamin D, vitamin E, vitamin K, K1 and K2 (i.e., MK-4, MK-7), folic acid, biotin, choline, their derivative or combinations thereof.

Minerals

Minerals may include boron, calcium, chromium, copper, iodine, iron, magnesium, manganese, molybdenum, nickel, phosphorus, potassium, selenium, silicon, tin, vanadium, zinc, amino acid chelated minerals, yeast cell wall chelated minerals, or combinations thereof.

Amino Acids

Amino acid may include for example alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, taurine, theanine, or combinations thereof.

Antioxidants

Antioxidant may include astaxanthin, carotenoids, coenzyme Q10 (“CoQ10”), flavonoids, glutathione, Goji (wolfberry), hesperidin, lacto-wolfberry, lignan, lutein, lycopene, polyphenols, selenium, vitamin A, vitamin C, vitamin E, zeaxanthin, or combinations thereof

Herbal Actives

The oral health chewable may include natural herbal extracts and powders. Herbal extracts and powders are either ground or extracted from flowers, leaves, bark, stems, roots, shrubs and trees. The extracts are dried to yield powders. The effectiveness herbal extracts and powders lies in their high concentrations of active beneficial oral health components. When used in the oral health chewable, the herbal extracts and powders are absorbed deep into gum tissue by physical action of chewing and through absorption through the mucous membranes where their potent antibacterial properties provide valuable benefits in the treatment of gum disease.

The source of phytonutrients may be flavonoids, allied phenolic compounds, polyphenolic compounds, terpenoids, alkaloids, sulphur-containing compounds, polysaccharides, flavone, flavonoids, quinone, or combinations thereof.

Essential Oils (EOs)

Essential oils (EOs) are distilled liquids extracted from flowers, leaves, bark, stems, roots, shrubs and trees. The effectiveness of EOs lies in their exceptional ability to penetrate human tissue and provide therapeutic benefits. EOs contain aromatic molecules that rapidly absorb through the oral mucous membranes and travel through the body through the circulatory system. When used in herbal dental care, EOs are absorbed deep into gum tissue and oral mucous membranes where their potent antibacterial properties provide valuable benefits in the treatment of gum disease. EOs have the additional function of providing flavor for the oral health chewable, Some EOs used for the oral health chewable are the following:

Red Thyme. The oil from this herb has been used extensively in medicine as a antiseptic and disinfectant. EOs from red thyme have antibacterial, antifungal, anti-inflammatory, antimicrobial and stimulating properties that help treat oral inflammation and infection.

Cinnamon Bark. The high aldehyde content of this oil results in it being an antimicrobial and antiseptic. Cinnamon has a tannins concentration resulting in cinnamon bark being an astringent. Astringents contract, firm and strengthen oral tissue, reduce surface inflammation and irritation, and create a protective barrier from infection.

Eucalyptus. Eucalyptus has antibacterial, antifungal, antiseptic and stimulant properties, which boost circulation and speed up the healing process and combats oral infection and mouth ulcers.

Lavender. The EO from this highly aromatic flower offers antibacterial, anti-inflammatory, antimicrobial, antiseptic and stimulant properties that result in the EO being a natural dental remedy, making it a strong ally for good oral health. Lavender is effective against bad breath (halitosis), helps heal damaged tissue and provides soothing relief from mouth pain.

Peppermint. The EO from this plant provides antibacterial, anti-inflammatory, antifungal, antimicrobial, antiseptic, sedative and stimulant properties. Peppermint EU used in the oral health chewable offers protection from oral infection, pain relief and enhanced blood circulation which leads to increased healing rates.

Tea tree. Tea tree (Melaleuca altemifolia) is a potent antimicrobial EO which fights the bacteria that cause tooth decay and gum disease. It is often used in combination with other natural herbs, such as rosemary, chamomile, echinacea, aloe and fresh mint and when used in the oral health chewable imparts anti-cavity and anti-gingivitis properties to the chewable.

Traditional Chinese Medicine (TCM) Herbals

In one embodiment, the herbal composition comprises a TCM herb or formulation. The TCM herb or formulation may have anti-inflammatory, anti-microbial, anti-bacteria, anti-viral, pain relief, antiseptic, analgesic, salivary promoting, anti-ulcer activities, or a combination thereof.

The herbal composition may include extract, powders or a combination. The extract may be water extract, ethanol extract, water/ethanol extract or other organic solvent extract.

The herbal powder may be derived from various plant parts including, for example, roots, leaves, stems, fruits, flowers, nuts, seeds, bulbs or a combination thereof.

Example TCM herbs include Zanthoxylum nitidum (Roxb.), Yan Husuo ((Corydalis yanhusuo W. T. Wang ex Z. Y. Su et C. Y. Wu), Qingfengteng (Sabia japonica Maxim.), Rehmannia glutinosa, Mulberry leaf, Guan Gui (cortex cinnamomic), Mentha (including for example, mint, peppermint or spearmint), Centella asiatica (L.) Urban, Pueraria lobate, Lophatherum gracile, Scutellaria baicalensis Georgi, Sarcandra glabra (Thunb.) Nakai, honeysuckle, Imperata cylindrica Beauv.var. major (Nees), C. E. Hubb., wild chrysanthemum, chamomile, licorice, ginseng, Ganoderma Lucidum Karst, Ribes nigrum L, Gingko, green tea, pearl powder, powder of mother of pearl, ginger, or a combination thereof.

Zanthoxylum nitidum (Roxb.) helps to relieve pain and promote blood circulation. Yan Husuo ((Corydalis yanhusuo W. T. Wang ex Z. Y. Su et C. Y. Wu) is a herb used to promote blood circulation. Qingfengteng (Sabia japonica Maxim.) is an herb used for anti-inflammatory applications including treating rheumatism and reducing swelling.

Panaxnotoginseng (Burk.) F. H. Chen has the effect of removing blood stasis and hemostasis, promoting blood circulation and relieving pain. The herb may be used in the disclosed composition to treating gum bleeding, bruises and swelling. Rehmannia glutinosa may be used to reduce inflammation, relieve pain, clearing heat and nourishing blood. Mulberry leaf may be used to clear the inflammation.

Guan Gui (cortex cinnamomic) may be used to promote blood circulation, relieve pain, reduce inflammation, detoxify and reduce swelling. Mentha (peppermint, spearmint) may be used to treat toothache, relieve coughing, cough, and reducing bleeding. Centella asiatica (L.) Urban may be used to reduce inflammation, reduce swelling and detoxification.

Pueraria lobate may be used to relieve muscles pain, reduce fever, reduce rashes, and treating diarrhea. Lophatherum gracile is an anti-inflammatory herb and may be used to treat fever, dehydration, tongue and mouth sore. It is also a diuretic.

Scutellaria baicalensis Georgi is an anti-inflammatory herb and may be used to detoxify and reduce. Scutellaria baicalensis has been shown to have antibacterial activities.

Sarcandra glabra (Thunb.) Nakai is an anti-inflammatory herb and may be used to reduce swelling and relieve pain. It has been shown to have antibacterial and anti-inflammatory properties.

Honeysuckle may be used to clear the inflammation, detoxify, and reduce fever. The herb has anti-inflammatory and anti-viral activities. Wild chrysanthemum may be used as an antipyretic and antipyretic herb.

Imperata cylindrica Beauv.var. major (Nees) C. E. Hubb. may be used as diuretic, blood coagulant, and immune booster. The research has shown that the herb has antibacterial effect. In one embodiment, the oral health composition comprises the extract or powder of the herbal root.

Ribes nigrum L may be used to promote gum health and protect teeth.

In one embodiment, the herbal composition provides pain relief, teeth cleaning and gum protection property. In one embodiment, the herbal composition comprises Zanthoxylum nitidum (Roxb.), Centella asiatica, mentha, or a combination thereof.

In one embodiment, the herbal composition provide pain relieve analgesic effect. In one embodiment, the herbal composition provides lastingly relieve toothache-related problems such as bleeding gums, red gums, and tooth sensitivity. In one embodiment, the herbal composition comprises Zanthoxylum nitidum (Roxb.), Yanhusuo, Guangui, or a combination thereof. These Chinese herbal medicines have the functions of rapid pain relief, swelling and hemostasis, and has the effects on inhibiting harmful bacteria in the oral cavity.

In one embodiment, the herbal composition provides a rapid pain relieve effect and can quickly relieve gum pain. In one embodiment, the herbal composition provides platelet aggregation and strengthen gum tissues against inflammation. In one embodiment, the herbal composition promotes microcirculation.

In one embodiment, the herbal composition provides pain relief and repairing function to the tooth. reducing swelling and hemostasis, clearing heat and regenerating, and deeply improving oral microbiota population.

In one embodiment, the herbal composition comprises Zanthoxylum nitidum (Roxb.), Yanhusuo, Qingfengteng, Rehmannia glutinosa, Mulberry leaf, or a combination thereof.

In one embodiment, the herbal composition can detoxify and eliminate swelling and remove harmful bacteria, inhibit bleeding and repair wounds, relieve blood stasis and relieve pain, restore gingival teeth and solid teeth layer by layer, and balance pH value to reshape good microbiota population.

In one embodiment, the herbal composition comprises ginger. Ginger has the functions of anti-inflammatory and fresh breath. In one embodiment, the disclosed composition comprises gingerols from about 0.001% to about 3%.

In one embodiment, the oral health composition is substantially void of sugar, fluorine, artificial color, fluorine, and artificial preservative. In one embodiment, the oral health composition contains active calcium and xylitol to nourishes the gums, increases calcium and strengthens teeth.

In one embodiment, the oral health composition comprises Dandelion, Isatisis (Radix Isatis), Scutellaria baicalensis, Radix Isatidis, or a combination thereof. In one embodiment, the oral composition comprises Dandelion Root, Radix Isatis, Corydalis bungeana, Baikal Skullcap, or a combination thereof. In one embodiment, the ratio of dandelion root, Isatisis (Radix Isatis), Corydalis bungeana, and Baikal Skullcap may be 4:1.5:1:1.5.

In one embodiment, the oral health composition comprises watermelon frost, a TCM herbal formulation made from watermelon skin and skin nitrate.

In one embodiment, the oral health composition comprises Yunnan Baiyao active ingredients. The composition is used to reduce gum problems (bleeding gums, gum pain), repairs mucosal damage, nourishes gums, and improves periodontal health.

In one embodiment, the herbal composition comprises notoginseng, musk, bovis calculus sativus, snack gallbladder. The composition has the effect of anti-inflammation, detoxify, promote blood circulation, reduce swelling and relieve pain.

In one embodiment, the herbal composition comprises honeysuckle, scutellaria baicalensis, and forsythia. It has the functions of reducing swelling, anti-inflammation and detoxifying.

In one embodiment, the herbal composition comprises loptatherum gracile and mint.

In one embodiment, the herbal composition comprises honeysuckle and wild chrysanthemum.

In one embodiment, the herbal composition comprises while chrysanthemum, chamomile, and lmperata cylindrinca.

In one embodiment, the herbal composition comprises radix rehmanniae, fructus corni officinalis, radix dioscoreae oppositae, alismatis rhizoma, sclerotium poriae cocos and cortex moutan radicis.

Tripterygium wilfordii is a native plant that grows in many parts of China and Burma. It has been commonly used in the treatment of a wide spectrum of autoimmune and inflammatory diseases.

In one embodiment, the herbal composition comprises asparagus root, tuber ophiopogonis japonici, radix scrophulariae ningpoensis, flos Lonicerae japonicae and radix glycyrrhizae uralensis.

In one embodiment, the herbal composition comprises radix ligustici chuanxiong, radix ginseng, radix paeoniae lactiflorae, semen persicae and radix et caulis jixueteng.

In one embodiment, the herbal composition comprises Vietnamese sophora root, bistort rhizome, north valerianaceae, cortex dictamni, Prunella vulgaris L and Dioscorea bulbifera.

In one embodiment, the herbal composition can modulate immune reactions, inhibit production of inflammatory cytokines, suppress tumor cell proliferation and reduce the incidence of squamous cell carcinoma.

In one embodiment, the herbal composition comprises a TCM herb with antimicrobial activity. Example TCM herbs include without limitation Fructus armeniaca mume, Porphyromonas gingivalis, including Cortex magnolias officinalis, Cortex phellodendri, Flos caryophylli, Flos lonicerae japonicae, Fructus armeniaca mume, Fructus forsythias suspensae, Herba cum radice violae yedoensitis, Herba menthae haplocalycis, Pericarpium granati, Radix et rhizoma rhei, Radix gentianae, Ramulus cinnamomi cassia and Rhizoma cimicifugae. In one embodiment, the herbal composition comprises Cortex phellodendri. In one embodiment, the herbal composition comprises Radix et rhizoma rhei. In one embodiment, the herbal composition comprises Fructus armeniaca mume.

In one embodiment, the herbal composition comprises Liangmianzheng (Zanthoxylum nitidum (Roxb.) DC).

In one embodiment, the herbal composition comprises Aloe Vera. Aloe vera has a soothing and anti-inflammatory effect. While not directly addressing any particular oral problem, its anti-inflammatory effects addresses the swelling brought on by many oral health issues.

In one embodiment, the herbal composition comprises Calendula officinalis. Calendula officinalis has soothing, anti-inflammatory properties that help heal irritated, tender gums. It also has a mild antiseptic action. Similar to aloe vera in not directly addressing any particular oral problem, its anti-inflammatory and antiseptic effects address the swelling brought on by many oral health issues.

In one embodiment, the herbal composition comprises echinacea. Extracts of echinacea are immune stimulating, which helps combat infection; it is anti-inflammatory and antiviral.

In one embodiment, the herbal composition comprises Goldenseal. Goldenseal (Hydrastis canadensis) has anti-inflammatory, antiviral and antibiotic properties; it's good for healing and preventing gum problems.

In one embodiment, the herbal composition comprises grapefruit. Grapefruit seed extract and freeze-dried pulp powders, made from the seeds and pulp of grapefruit, has antiseptic and antibacterial properties to fight infection.

In one embodiment, the herbal composition comprises horsetail. The extracts of horsetail are high in concentration in silica, vitamin E, selenium and other minerals. When used in the oral health chewable, it can help in coagulation, decrease bleeding and heal oral infections.

In one embodiment, the herbal composition comprises myrrh. Myrrh (Commiphora molmol) has antimicrobial and astringent properties that help to tighten the gums. When used in the oral health chewable it aids in healing bleeding gums and mouth ulcers, and helps fight the bacteria that cause tooth decay and gum disease.

In one embodiment, the herbal composition comprises neem. Neem (Azadirachta indica), long considered a top antibacterial herb in Ayurvedic (a system of medicine with historical roots in the Indian subcontinent) healing, helps prevent plaque. When used in the oral health chewable it helps prevent the diseases associated with plaque buildup including gingivitis and dental caries.

In one embodiment, the herbal composition comprises Oregon grape root. Oregon grape root (Mahonia spp.) has a high concentration of antimicrobial compounds and astringent properties; which, when used in the oral health chewable results to prevent and heal gum problems.

In one embodiment, the herbal composition comprises sage. Sage (Salvia officinalis) has strong astringent properties, making it a favorite herb for tightening the gums and soothing a sore mouth.

In one embodiment, the herbal composition comprises white oak bark. White oak bark (Quercus alba) is a strong astringent that is helpful for healing swollen, tender and bleeding gums and mucous membranes, and has a clotting and antiseptic effect.

In one embodiment, the herbal composition comprises turmeric. In one embodiment, the herbal composition comprises curcumin. Turmeric, especially curcumin is an effective natural alternative for the management of oral health problems which is specifically documented in the following: 1. Clinical efficacy of turmeric use in gingivitis: A comprehensive review, Stoyell K A, Mappus J L, Gandhi M A, Complement Ther Clip Pract. 2016 November; 25: 13-17). 2. Efficacy of curcumin in the treatment of chronic gingivitis: a pilot study. Muglikar, S., Patil, K. C., Shivswami, S., and Hegde, R., Oral Health and Preventive Dentistry. 2012; 11(1):81-86) 3. comparison of effectiveness of curcumin with triamcinolone acetonide in the gel form in treatment of minor recurrent aphthous stomatitis: A randomized clinical trial., Deshmukh R A, Bagewadi A S, Int J Pharm Investig. 2014 July; 4(3):138-41). 4. Oral care compositions, Brading et al., U.S. Pat. No. 8,916,139.

In one embodiment, the herbal composition comprises tea. Chen et al. further indicated the abilities of epigallocatechin-3-gallate, luteolin, apigenin, myricetin, quercetin, and cyanidin in inhibiting retinal pigment epithelial cells in “Effects of the vegetable polyphenols epigallocatechin-3-gallate, luteolin, apigenin, myricetin, quercetin, and cyanidin in primary cultures of human retinal pigment epithelial cells” (Molecular Vision, 2014, 20:242-258). More studies indicated the abilities of epigallocatechin-3-gallate in inhibiting the proliferation of aortic smooth cells: (Z. Shu, M. Yu, G. Zeng, X. Zhang, L. Wu, X. Tan, 2014, “Epigallocatechin-3-gallate inhibits proliferation of human aortic smooth muscle cells via up-regulating expression of mitofusin,” European Journal of Cell Biology, 93:137-144; P. L. Liu, J. T. Liu, H. F. Kuo, I. W. Chong, and C. C. Hsieh, “Epigallocatechin Gallate Attenuates Proliferation and Oxidative Stress in Human Vascular Smooth Muscles Cells Induced by Interleukin-1.beta. via Heme Oxygenase-1,” 2014, Mediators of Inflammation, Article ID 523684.

Polyphenols

Fibroblasts play crucial roles during wound healing. When the existence of a wound is detected, fibroblasts will be activated to become polygonal phenotype as discussed by Li Y C et al. in “Pearl extract enhances the migratory ability of fibroblasts in a wound healing model” (Pharm. Biol., 2013, 51:289-297). When the wound is healing, fibroblasts will proliferate and move to the location of the wound for repairing as discussed by Khovidhunkit, S. O. et al. in “In vitro study of the effects of plaunotol on oral cell proliferation and wound healing” (J. Asian Nat. Prod. Res., 2011, 13:149-159). The increased quantity of fibroblasts improved the condition of wound healing in experimental models as discussed by Lamme E. N. et al. in “Higher numbers of autologous fibroblasts in an artificial dermal substitute improve tissue regeneration and modulate scar tissue formation” (J. Pathol., 2000, 1900:595-603).

In one embodiment, the herbal composition comprises plant polyphenol effective in improving the proliferation of fibroblasts. In one embodiment, the polyphenol comprises a citrus polyphenol. In one embodiment, the citrus polyphenol includes delphinidin, pelargonidin, peonidin, malvidin, cyaniding, narirutin, naringin, hesperidin, and neohesperidin.

Probiotics

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects. These probiotic features are strain-dependent, even among bacteria of the same species. Therefore, it is important to find those strains that have a better performance in all probiotic requirements. In one embodiment, the probiotic can adhere to and colonize surfaces in the oral cavity.

Probiotics have many positive influences in creating better oral health. Probiotics have both direct and indirect interactions. The advantages of direct interactions are many. Basically, probiotics help in binding oral microorganisms to proteins & biofilm formation. They fight against plaque formation and on its complex ecosystem by compromising and intervening with bacterial attachments. Through its direct interactions, probiotics compete with oral microorganisms of substances available. This process is the involvement of metabolism of substrate. Probiotics produce chemicals to inhibit oral harmful bacteria that damage oral hygiene. On the other hand, the indirect interactions of probiotics are effective in the process of removing harmful bacteria and stabilizing normal conditions. Probiotics modulate and systematize immune function on local community as well as non-immunologic defense mechanisms. Probiotics have the ability to regulate permeability and also to develop colonies in oral microflora with less pathogenic species. Probiotics have proved to be effective in curing diseases such as dental caries, periodontal diseases, halitosis and candidiasis.

Example probiotics that may be useful in the currently application include S. salivarius, L. rhamnosus GG, L. acidophilus, L. casei, L. reuteri, Bifidobacterium DN-173010, Propionibacterium, Freudenreichil ssp., Shermanil J S, L. paracasei, L. johnsonil, W. cibaria, L. casei Shirota, or a combination thereof. The biological advantage may include reduction of volatile sulfur compounds (VSC), streptococcus mutant (S. mutant) inhibition, reduction of S. mutant levels, reduction of levels of caries pathogens, reduction of high yeast counts, inhibition of S. mutants adhesion to salivary pellicle, inhibition of production of VSC, or a combination thereof.

In one embodiment, the probiotics include Lactobacillus plantarum CECT 7481, Lactobacillus brevis CECT 7480, their mutant or a combination thereof. “Lactobacillus plantarum CECT 7481” is understood as the Lactobacillus plantarum strain deposited in the Spanish Type Culture Collection under the accession number CECT 7481. “Lactobacillus brevis CECT 7480” is understood as the Lactobacillus brevis strain deposited in the Spanish Type Culture Collection under the accession number CECT 7480. Mutant or derivative microorganisms may be obtained by techniques known in the state of the art using the deposited strain as starting material, such mutants or derivatives at least retaining the herein described relevant features and advantages of the starting strain.

Lactobacillus plantarum CECT 7481 and Lactobacillus brevis CECT 7480 display a significant inhibitory activity against a broad number of pathogens of the oral cavity that are implicated in the development of oral disorders, such as gingivitis, periodontitis, caries and halitosis, while displaying minimal antagonism against common commensal strains of the human oral flora.

A probiotic L. reuteri ATCC 55730 strain and has demonstrated to reduce gingivitis in a clinical trial (Twetman S, et al. “Short-term effect of chewing gums containing probiotic Lactobacillus.sub.--reuteri on the levels of inflammatory mediators in gingival crevicular fluid”. Acta Odontol Scand, 2009, vol. 67, p. 19-24). This same strain, L. reuteri ATCC 55730, has been reported to exert a strong antagonistic activity against cariogenic

Streptococcus salivarius K12 is another commercial probiotic intended for use in the oral cavity. S. salivarius K12 was isolated from the saliva of a healthy child and has been shown to perform in vitro antimicrobial activity against various bacterial species incriminated in the etiology of halitosis (Burton J P, et al., “Preliminary study of the effect of probiotic Streptococcus salivarius K12 on oral malodor parameters”. J Appl Microbiol, 2006, vol. 100, p. 754-764). However, the beneficial effects of this strain are limited to the amelioration of halitosis symptoms.

By antagonizing microorganisms that are implicated in pathological conditions in the oral cavity, such as Streptococcus mutans, Porphyromonas gingivalis, Treponema denticola, Prevotella denticola and Fusobacterium nucleatum, these strains have the effect of altering the oral microbiological profile to a healthier profile, thereby benefiting oral health conditions.

Probiotic may include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

The term “effective amount” as used herein is the amount of colony forming units (cfu) for each strain in the composition that is high enough to significantly modify the condition to be treated in a positive way but low enough to avoid serious side effects (at a reasonable benefit/risk ratio), within the scope of sound medical judgment. An effective amount of said probiotic microorganism will be determined by the skilled in the art and will vary with the particular goal to be achieved, the age and physical condition of the patient being treated, the severity of the underlying disorder, and the final formulation. For instance, in oral health products, the strain or strains are present in an amount from about 10.sup.5 cfu/g to about 10.sup.12 cfu/g, such as in an amount from about 10.sup.7 cfu/g to about 10.sup.11 cfu/g. The term “colony forming unit” (“cfu”) is defined as number of bacterial cells as revealed by microbiological counts on agar plates. In a particular embodiment, the composition of the application is an oral care product comprising between 10.sup.7-10.sup.10 cfu/g.

In one embodiment, the composition may include probiotic strains in an amount ranging from 10.sup.5 and 10.sup.12 cfu/g. In a particular embodiment, the composition of the application is a dietary supplement comprising between 10.sup.7-10.sup.10 cfu/g.

Prebiotics

Prebiotics may enhance the growth and activity of beneficial organisms and simultaneously suppress the growth and activity of potentially deleterious bacteria, therefore, modifies the balance of the intestinal micro-flora or change microbial population density.

Useful prebiotics may include Lactose, Inulin, Fructo oligosacccharides, Galacto oligosaccharides and Xylo oligosaccharides. Prebiotics are naturally found plenty in certain fruits like bananas, asparagus, garlic, tomato and onion wheat.

Prebiotics when combined with probiotics have many advantages. In one embodiment, prebiotics selectively stimulate the growth of probiotics, which may be dose and strain dependent. Prebiotics serve as a selective growth substrate for the probiotics strain during fermentation, during the period of storage, or during its passage through the gut.

Prebiotic may include for example acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

Polishing Agents

The polishing agent may include hydrated silica, calcium, bentonite, kaolin clay, pearl powder, the powder of mother of pearl, nutshell powder, salt, or a combination thereof.

An oral or pharmaceutically acceptable abrasive or polishing agent can include one or a mixture of abrasives such as for example, calcium carbonate, sodium metaphosphate, potassium metaphosphate, tricalcium phosphate, di-hydrated dicalcium phosphate, calcined alumina and siliceous materials, bentonite clay, kaolin clay or combinations thereof. Example abrasives include calcium carbonate, siliceous materials, such as silica and more such as hydrated silica, bentonite clay, kaolin clay or combinations thereof. More such as abrasives include a mixture of calcium carbonate, bentonite clay, kaolin clay and hydrated silica. The abrasive content the oral care composition embodiments can be in the range (weight %) of from about of 1% to about 80%, from about 35% to about 50%.

In one embodiment, the polishing agent may be finely divided silica, calcium carbonate, tricalcium phosphate, dicalcium phosphate, insoluble sodium metaphosphate, or a combination thereof.

Oxidizing Agents

An oral or pharmaceutically acceptable oxidizing agent can include one or a mixture of oxidizing agents including peroxide compounds or its derivatives, such as, for example, hydrogen peroxide, peroxydiphosphate, urea peroxide, metal peroxides such as calcium peroxide, sodium peroxide, stronthium peroxide, magnesium peroxide, and the salts of perborate, persilicate, perphosphate and percarbonate such as sodium perborate, potassium persilicate, sodium percarbonate, or a combination thereof.

The oxidizing agent content the oral care composition embodiments can be in the range of from about of 1 w % to about 35 w %, from about 0.5 w % to about 10%, about 5 w % to 14 w %.

Peroxide Stabilizers

An oral or pharmaceutically acceptable bleaching agent or peroxide stabilizer can include one or a mixture of such agents, such as, for example, ethylenediamineteraacetic acid, disodium salt; ethylenediamineteraacetic acid, tetrasodium salt; ethylenediaminetetraacetic acid, calcium disodium salt; etidronic acid; citric acid; gluconic acid; sodium citrate; sodium gluconate; sodium phosphate; disodium phosphate; trisodium phosphate; tetrapotassium pyrophosphate; sodium tripolyphosphate as well as sodium stannate and potassium stannate (tin can be a or peroxide stabilizer). The peroxide stabilizer content can be in the range of from about of 0.01 w % to about 2 w %, from about 0.05 w % to about 0.3 w %.

Anti-Caries Agents

Embodiments of the present disclosure can also include sources of fluoride ions, or fluorine-providing compounds useful, for example, as anti-caries agents. An oral or pharmaceutically acceptable fluoride source can include one or a mixture of fluoride source. In one embodiment, the anti-caries agent comprises fluoride salts, such as soluble alkali metal and alkaline earth metal salts. Examples include sodium fluoride, potassium fluoride, ammonium fluoride, calcium fluoride, copper fluorides (such as cuprous fluoride), zinc fluoride, barium fluoride, sodium fluorosilicate, ammonium fluorosilicate, sodium fluorozirconate, sodium monofluoro-phosphate, aluminum mono- and di-fluorophosphate, fluorinated sodium calcium pyrophosphate, alkali metal and tin fluorides (such as sodium and stannous fluoride), sodium monofluorophosphate, and mixtures thereof. The anti-caries agent in the oral care composition may be at concentrations ranging from about 0.005 w % to about 3 w %.

Tooth Hardening Agents

In one embodiment, the oral health composition comprises a tooth hardening agent. In one embodiment, the tooth hardening agent includes soluble alkali metal fluorides, sodium fluoride, potassium fluoride, copper fluoride, tin fluorides, ammonium fluorosilicate, sodium fluorozirconate, ammonium fluorozirconate, sodium monofluorophosphate, aluminum fluorophosphates (mono-, di- and tri-), fluorinated sodium calcium pyrophosphate, sodium monofluorophosphate, or a combination thereof,

Anti-Tartar Agent

In one embodiment, the oral health composition comprises an anti-tartar agent. In one embodiment, the anti-tartar agent includes polyphosphates, alkali metal tripolyphosphates, alkali metal pyrophosphates, sodium pyrophosphate, or a combination thereof.

Anti-Calculus Agent

In one embodiment, the oral health composition comprises an anti-calculus agent. In one embodiment, the anti-calculus agent includes an azacycloalkane diphosphonic compound, azacycloheptane diphosphonic acid and salts thereof, synthetic anionic polymeric polycarboxylates, copolymers of maleic acid or maleic anhydride with vinyl methyl ether, their salts, derivatives, or a combination thereof.

Remineralizing Agents

Calcium glycerophosphate is a dental agent capable of remineralizing enamel. In certain embodiments, remineralizing agent may include a phosphate compound, a calcium compound, a calcium phosphate compound, hydroxyapatite and a caseinate. The phosphate compound may be a monobasic phosphate compound, a dibasic phosphate compound, a tribasic phosphate compound, calcium glycerophosphate, or combinations thereof.

Desensitizing Agents

An oral or pharmaceutically acceptable agent used to diminish teeth sensitivity may be included in the disclosed compositions. In some embodiments, the desensitizing agents may include strontium chloride, potassium nitrate, potassium citrate, or a combination thereof. The desensitizing agent in the disclosed composition may be in the range of from about of 0.1 w % to about 5 w %. In one embodiment, the oral care composition comprises potassium nitrate.

pH Adjusting Agents

An oral or pharmaceutically acceptable pH adjuster may include sodium bicarbonate, sodium hydroxide, ammonium hydroxide, calcium glycerophosphate, triethanolamine (an organic compound composed of a tri-alcohol & an amine), or a combination thereof. In some embodiments, sodium bicarbonate can be at concentrations from about 5 w % to about 99.5 w %, from about 10 w % to about 80 w %. In one embodiment, when the pH-adjusting agents are in powder form, it may be from about 90 w % to about 99.5 w %. In one embodiment, a combination of sodium bicarbonate and sodium hydroxide may be used. In one embodiment, sodium bicarbonate can be at concentrations from about 15 w % to about 79 w % and sodium hydroxide may be at concentrations from about 1 w % to about 5 w %.

Breath Freshen Agents

The disclosed composition may include an oral or pharmaceutically acceptable anti-bad-breath agent for treating bad breath (e.g. halitosis). In one embodiment, the freshen agent may cetylpyridinium chloride (CPC), zinc compounds (e.g., zinc chloride, zinc citrate and Zinc gluconate), chlorhexidine, or a combination thereof. In some embodiment, the anti-bad-breath agents content in the oral care composition may be in the range of from about of 0.05 w % to about 5 w %, from about 0.075 w % to about 2 w %, or any number in between.

Chelating Agents

The disclosed composition may include an oral or pharmaceutically acceptable chelating agent. Examples include sodium tripolyphosphate, ethylenediamine tetracetic acid (“EDTA”) and its salts (e.g., tetrasodium EDTA and calcium EDTA), or a combination thereof. In some embodiments, the chelating agents may be at concentrations of from about 0.3 w % to about 30 w %. In one embodiment, the chelating agent comprises sodium tripolyphosphate.

In one embodiment, the chelating agent may be ethylenediamine tetraacetic acid (EDTA), ethylene glycol tetraacetic acid (EGTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ArPA), citric acid, acetic acid and acceptable salts thereof, and any combinations thereof.

In one embodiment, the EDTA salt may be diammonium EDTA, disodium EDTA, dipotassium EDTA, triammonium EDTA, trisodium EDTA, tripotassium EDTA, tetrasodium EDTA, tetrapotassium EDTA, calcium disodium EDTA, or combinations thereof.

In one embodiment, the chelating agent is may be phosphates, pyrophosphates, tripolyphosphates, hexametaphosphates, or a combination thereof.

In one embodiment, the chelating agent may be a polyamine selected from cyclam (1,4,7,11-tetraazacyclotetradecane), N—(C.sub.1-C.sub.30 alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N-{5-[Acetyl(hydroxy)amino]pentyl}-N-[5-({4-[(5-aminopentyl)(hydroxy)am-ino]-4-oxobutanoyl}amino)pentyl]-N-hydroxysuccinamide, or N′-[5-(Acetyl-hydroxy-amino)pentyl]-N-[5-[3-(5-aminopentyl-hydroxy-carbam-oyl) propanoylamino]pentyl]-N-hydroxy-butane diamide), desferrioxamine B, desferoxamine B, DFO-B, DFOA, DFB, desferal, deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

In one embodiment, the chelating agent may be a EDTA-4-aminoquinoline conjugate selected from ([2-(Bis-ethoxycarbonylmethyl-amino)-ethyl]-{[2-(7-chloro-quinolin-4-ylam-ino)-ethylcarbamoyl]-methyl}-amino)-acetic acid ethyl ester, ([2-(Bis-ethoxycarbonylmethyl-amino)-propyl]-{[2-(7-chloro-quinolin-4-yla-mino)-ethylcarbamoyl]-methyl}-amino)-acetic acid ethyl ester, ([3-(Bis-ethoxycarbonylmethyl-amino)-propyl]-{[2-(7-chloro-quinolin-4-yla-mino)-ethylcarbamoyl]-methyl}-amino)-acetic acid ethyl ester, ([4-(Bis-ethoxycarbonylmethyl-amino)-butyl]-{[2-(7-chloro-quinolin-4-ylam-ino)-ethylcarbamoyl]-methyl}-amino)-acetic acid ethyl ester, ([2-(Bis-ethoxymethyl-amino)-ethyl]-{[2-(7-chloro-quinolin-4-ylamino)-eth-ylcarbamoyl]-methyl}-amino)-acetic acid ethyl ester, ([2-(Bis-ethoxymethyl-amino)-propyl]-{[2-(7-chloro-quinolin-4-ylamino)-et-hylcarbamoyl]-methyl}-amino)-acetic acid ethyl ester, ([3-(Bis-ethoxymethyl-amino)-propyl]-{[2-(7-chloro-quinolin-4-ylamino)-ethylcarbamoyl]-methyl}-amino)-acetic acid ethyl ester, ([4-(Bis-ethoxymethyl-amino)-butyl]-{[2-(7-chloro-quinolin-4-ylamino)-eth-ylcarbamoyl]-methyl}-amino)-acetic acid ethyl ester.

In one embodiment, the chelating agent may be a tetrasodium salt of iminodisuccinic acid. In one embodiment, the chelating agent is a salt of poly-asparatic acid. In one embodiment, the chelating agent is a tetra sodium salt of L-glutamic acid N,N-diacetic acid. In one embodiment, the chelating agent is a natural chelator selected from citric acid, phytic acid, lactic acid, acetic acid and their salts and curcumin.

Occlusive Agents

The disclosed composition may include an oral or pharmaceutically acceptable occlusive agent. Examples include petrolatum, mineral oil, beeswax, silicone oil, lanolin and oil-soluble lanolin derivatives, saturated and unsaturated fatty alcohols (such as behenyl alcohol), hydrocarbons (such as squalene), and various animal and vegetable oils (such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil or sunflower seed oil). The occlusive agent may be in the range of from about of 1 ww % to about 35 w %, from about 1 w % to about 10 w %. In one embodiment, the occlusive agent comprises coconut oil.

In an embodiment, the oral health semi-chewable composition may be in a chewy solid or semi-solid form having a shape selected from the group consisting of a bar, a cylinder, a sphere, a cube, a star, a prism, a disc, or combinations thereof.

In other aspects the application provides an oral health composition that contains pharmaceutically acceptable excipients. Selection of the excipients and the most appropriate methods for formulation in view of the particular purpose of the composition is within the scope of the person skilled in the art of pharmaceutical technology.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present application. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The term “pharmaceutically acceptable” as used herein refers to compounds, materials, compositions, and/or dosage forms which are, within the scope of sound medical judgement, suitable for use in contact with the tissues of a subject (either a human or non-human animal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical texts.

Another aspect of the application provides a cosmetical composition that contains the composition of the application together with cosmetically acceptable excipients. In preventing and/or treating oral disorders caused by pathologic bacteria, the composition of the application is useful for the amelioration and/or prevention of the symptoms produced by these disorders. Such symptoms include, but are not limited to, bad breath and stained teeth.

The term “cosmetically acceptable” refers to compounds, materials, compositions, and/or dosage forms which are, within the scope of sound medical judgement, suitable for use in contact with human skin without undue toxicity, incompatibility, instability and allergic response, among others. Each “cosmetically acceptable” carrier, excipient, etc., must also be “acceptable” in the sense of being compatible with the other ingredients of the cosmetic formulation. Suitable carriers, excipients, etc. for cosmetic formulations can be found in standard texts.

### EXAMPLES

### Example 1

General Procedure for Non-Dissolving Gum Base Manufacture

1. Cooking and Purifying the Base

The gum base is cooked in kettles at 116 degrees Celsius until it has melted into a thick syrup. To purify it, the gum base is passed through screens and placed in a high-speed centrifuge before re-filtering it through finer screens.

2. Blending Additional Ingredients

The gum base is now ready for additives. The gum base is placed in kettles to be cooked, and additional ingredients are stirred in by large steel blades. First, extremely fine powdered non cariogenic sweeteners are blended with the herbal ingredients and fine powdered fluoride salt. The blend is added to the gum base. Essential Oils and antigingivitis agents are blended together and are added next, followed by wax, surfactants and plasticizers and lastly the vitamins. When the mixture is smooth enough, it is rolled out onto belts and cooled by being exposed to cold air.

3. Kneading and Rolling the Gum

The next step is kneading. For several hours machines gently pummel the mass of chewing gum until it is properly rubbery and smooth. At this point the whitening agents are added and blended into the gum mass. Large chunks are then chopped off the mass, to be flattened by rollers until they reach the proper thickness. During this process, the sheet of chewing gum is dusted with powdered sweetener to prepare it for cutting.

4. Cutting and Seasoning the Gum

A cutting machine first scores the sheet into the desired shape and size. The sheet is then put aside at the proper temperature and humidity to “season.”

### Example 2

General Procedure for Dissolving Chewable Gummy Base Manufacture

1. Preparation of Water Solution

The herbal ingredients, optionally fluoride salts, and are combined and added to warm water at 90° C.

2. Preparation of Gelling Agent

Gelling agent is mixed with non-cariogenic sweetener and added to the water solution. Soak with low heat and stirring until the gum dissolves. Strain through a fine sieve to remove foreign material.

3. Syrup Mixture Preparation

A syrup is created by mixing non-cariogenic sweetener, humectant, and water and heated to 124° C.

4. Preparation of the Gum Base

The syrup mixture is poured into the gum solution and mixed gently.

5. Addition of Actives

Essential oils, antigingivitis agents, waxes, surfactants and vitamins are added to the gum base with gentle stirring.

6. Molding

The gum base is added to molds and allowed to dry at 49° C. until a water content of 4-5% is reached.

7. Coating

Non-cariogenic sweetener and teeth whitener are blended together and are applied to the surface of the molded oral health chewable.

### Example 3: Non-Dissolving Gum Base Examples

### Example 3.1

| Ingredient | Percentage |
|---|---|
| Natural Rubber | 18 |
| Hydrogenated Soybean Oil | 0.9 |
| Lecithin | 0.2 |
| Vegetable Oil | 0.7 |
| Bees Wax | 7 |
| Carnauba Wax | 6 |
| Stannous Fluoride | 0.05 |
| Glycerol | 8 |
| Cetylpyridinium chloride | 0.01 |
| Xylitol | 20 |
| Maltitol | 15 |
| Isomalt | 10 |
| Essential Oil | 0.5 |
| Aloe vera | 2 |
| Peppermint Essential Oil | 0.5 |
| Sodium Perborate | 5 |
| Vitamin Premix | 0.14 |
| Calendula Extract | 4 |
| Myrrh Extract | 2 |
| Total | 100 |

### Example 3.2

| Ingredient | Percentage |
|---|---|
| Natural Rubber | 18 |
| Hydrogenated Soybean Oil | 0.9 |
| Lecithin | 0.2 |
| Vegetable Oil | 0.7 |
| Bees Wax | 7 |
| Carnauba Wax | 6 |
| Stannous Fluoride | 0.05 |
| Glycerol | 8 |
| Cetylpyridinium chloride | 0.01 |
| Psicose | 20 |
| Trehalose | 13 |
| Isomaltulose | 12 |
| Essential Oil | 0.5 |
| Aloe vera | 2 |
| Peppermint Essential Oil | 0.5 |
| Sodium Perborate | 5 |
| Vitamin Premix | 0.14 |
| Calendula Extract | 3 |
| Myrrh Extract | 2 |
| Sage Extract | 1 |
| Total | 100 |

### Example 3.3

| Ingredient | Percentage |
|---|---|
| Natural Rubber | 18 |
| Hydrogenated Soybean Oil | 0.9 |
| Lecithin | 0.2 |
| Vegetable Oil | 0.7 |
| Bees Wax | 7 |
| Carnauba Wax | 6 |
| Stannous Fluoride | 0.05 |
| Glycerol | 8 |
| Cetylpyridinium chloride | 0.01 |
| Psicose | 20 |
| Trehalose | 13 |
| Isomaltulose | 12 |
| Essential Oil | 0.5 |
| Aloe vera | 2 |
| Peppermint Essential Oil | 0.5 |
| Sodium Perborate | 5 |
| Vitamin Premix | 0.14 |
| Horsetail PWD | 3 |
| Oregon grape root Extract | 2 |
| White oak bark PWD | 1 |
| Total | 100 |

### Example 3.4

| Ingredient | Percentage |
|---|---|
| Natural Rubber | 18 |
| Hydrogenated Soybean Oil | 0.9 |
| Lecithin | 0.2 |
| Vegetable Oil | 0.7 |
| Bees Wax | 7 |
| Carnauba Wax | 6 |
| Stannous Fluoride | 0.05 |
| Glycerol | 8 |
| Cetylpyridinium chloride | 0.01 |
| Psicose | 20 |
| Tagatose | 13 |
| Xylitol | 12 |
| Essential Oil | 0.5 |
| Aloe vera | 2 |
| Peppermint Essential Oil | 0.5 |
| Sodium Perborate | 5 |
| Vitamin Premix | 0.14 |
| Calendula Extract | 3 |
| Myrrh Extract | 2 |
| Sage Extract | 1 |
| Total | 100 |

### Example 3.5

| Ingredient | Percentage |
|---|---|
| Natural Rubber | 18 |
| Hydrogenated Soybean Oil | 0.9 |
| Lecithin | 0.2 |
| Vegetable Oil | 0.7 |
| Bees Wax | 7 |
| Carnauba Wax | 6 |
| Stannous Fluoride | 0.05 |
| Glycerol | 8 |
| Hexetidine | 0.01 |
| Xylitol | 20 |
| Sorbitol | 13 |
| Mannitol | 12 |
| Essential Oil | 0.5 |
| Aloe vera | 2 |
| Peppermint Essential Oil | 0.5 |
| Sodium Perborate | 5 |
| hydroxy-alpha-sanhoo | 0.14 |
| Horsetail PWD | 3 |
| Oregon grape root Extract | 2 |
| White oak bark PWD | 1 |
| Total | 100 |

### Example 3.6

| Ingredient | Percentage |
|---|---|
| Natural Rubber | 18 |
| Hydrogenated Soybean Oil | 0.9 |
| Lecithin | 0.2 |
| Vegetable Oil | 0.7 |
| Bees Wax | 7 |
| Carnauba Wax | 6 |
| Stannous Fluoride | 0.05 |
| Glycerol | 8 |
| Hexetidine | 0.01 |
| Xylitol | 20 |
| Maltitol | 15 |
| Isomalt | 10 |
| Essential Oil | 0.5 |
| Aloe vera | 2 |
| Peppermint Essential Oil | 0.5 |
| Sodium Perborate | 5 |
| Vitamin Premix | 0.14 |
| Calendula Extract | 3 |
| Neem Extract | 3 |
| Total | 100 |

### Example 4: Dissolving Gum Base

### Example 4.1

| Ingredient | Percentage |
|---|---|
| Gum Arabic | 35 |
| Xylitol | 15 |
| Maltitol | 15 |
| Isomalt | 10 |
| Glycerol | 10 |
| Bees Wax | 3 |
| Carnauba Wax | 1.8 |
| Stannous Fluoride | 0.05 |
| Cetylpyridinium chloride | 0.01 |
| Lecithin | 0.2 |
| Cinnamon Essential Oil | 0.5 |
| Aloe Vera | 2 |
| Sodium Perborate | 0.3 |
| Vitamin Premix | 0.14 |
| Myrrh Extract | 5 |
| Sage Extract | 2 |
| Total | 100 |

### Example 4.2

| Ingredient | Percentage |
|---|---|
| Gum Arabic | 25 |
| Xylitol | 20 |
| Maltitol | 18 |
| Isomalt | 12 |
| Glycerol | 10 |
| Bees Wax | 3 |
| Carnauba Wax | 1.8 |
| Stannous Fluoride | 0.05 |
| Cetylpyridinium chloride | 0.01 |
| Lecithin | 0.2 |
| Mint Essential Oil | 0.5 |
| Chrysanthemum extract | 2 |
| Sodium Perborate | 0.3 |
| Vitamin Premix | 0.14 |
| Chamomile extract | 5 |
| Imperata cylindrinca extract | 2 |
| Total | 100 |

### Example 4.3

| Ingredient | Percentage |
|---|---|
| Gum Arabic | 20 |
| Xylitol | 25 |
| Sorbitol | 18 |
| Mannitol | 12 |
| Glycerol | 10 |
| Bees Wax | 3 |
| Carnauba Wax | 1.8 |
| Stannous Fluoride | 0.05 |
| Cetylpyridinium chloride | 0.01 |
| Lecithin | 0.2 |
| Cinnamon Essential Oil | 0.5 |
| Zanthoxylum nitidum (Roxb.) DC) | 2 |
| Sodium Perborate | 0.3 |
| Vitamin Premix | 0.14 |
| Calendula Extract | 5 |
| Horsetail PWD | 2 |
| Total | 100 |

### Example 4.4

| Ingredient | Percentage |
|---|---|
| Gum Arabic | 20 |
| Xylitol | 25 |
| Sorbitol | 15 |
| Mannitol | 12 |
| Glycerol | 10 |
| Bees Wax | 3 |
| Carnauba Wax | 1.8 |
| Stannous Fluoride | 0.05 |
| Chlorhexidine | 0.005 |
| Caffeic | 0.005 |
| Lecithin | 0.2 |
| Clove Essential Oil | 0.5 |
| Notoginseng | 2 |
| Sodium Perborate | 0.3 |
| Vitamin Premix | 0.14 |
| Grapefruit Powder | 10 |
| Total | 100 |

### Example 4.5

| Ingredient | Percentage |
|---|---|
| Gum Arabic | 35 |
| Psicose | 18 |
| Trehaolse | 15 |
| Isomaltulose | 10 |
| Glycerol | 8 |
| Bees Wax | 3 |
| Carnauba Wax | 1.8 |
| Stannous Fluoride | 0.05 |
| Chlorhexidine | 0.005 |
| Caffeic | 0.005 |
| Lecithin | 0.2 |
| Cinnamon Essential Oil | 0.5 |
| Aloe Vera | 1 |
| Sodium Perborate | 0.3 |
| Vitamin Premix | 0.14 |
| Echinacea extract | 4 |
| Goldenseal | 3 |
| Total | 100 |

### Example 4.6

| Ingredient | Percentage |
|---|---|
| Gum Arabic | 25 |
| Psicose | 25 |
| Trehaolse | 17 |
| Isomaltulose | 11 |
| Glycerol | 8 |
| Bees Wax | 3 |
| Carnauba Wax | 1.8 |
| Stannous Fluoride | 0.05 |
| Chlorhexidine | 0.005 |
| Caffeic | 0.005 |
| Lecithin | 0.2 |
| Lavender Essential Oil | 0.5 |
| Honeysuckle extract | 1 |
| Sodium Perborate | 0.3 |
| Vitamin Premix | 0.14 |
| scutellaria baicalensis extract | 4 |
| Forsythia extract | 3 |
| Total | 100 |

### Example 4.7

| Ingredient | Percentage |
|---|---|
| Gum Arabic | 15 |
| Psicose | 33 |
| Trehaolse | 15 |
| Isomaltulose | 15 |
| Glycerol | 8 |
| Bees Wax | 3 |
| Carnauba Wax | 1.8 |
| Stannous Fluoride | 0.05 |
| Chlorhexidine | 0.005 |
| Caffeic | 0.005 |
| Lecithin | 0.2 |
| Thyme Essential Oil | 0.5 |
| Dandelion roots extract | 1 |
| Sodium Perborate | 0.3 |
| Vitamin Premix | 0.14 |
| Radix Isatidis extract | 3 |
| Isatisis (Radix Isatis) extract | 3 |
| Scutellaria baicalensis extract | 1 |
| Total | 100 |

### Example 4.8

| Ingredient | Percentage |
|---|---|
| Gum Arabic | 15 |
| Resistant Starch | 2 |
| Psicose | 31 |
| Trehaolse | 15 |
| Isomaltulose | 15 |
| Glycerol | 8 |
| Bees Wax | 3 |
| Carnauba Wax | 1.8 |
| Stannous Fluoride | 0.05 |
| Chlorhexidine | 0.005 |
| Caffeic | 0.005 |
| Lecithin | 0.2 |
| Tea Tree Essential Oil | 0.5 |
| Zanthoxylum nitidum (Roxb.) | 1 |
| Sodium Perborate | 0.3 |
| Vitamin Premix | 0.14 |
| Centella asiatica | 3 |
| mentha | 3 |
| Yanhusuo | 1 |
| Total | 100 |

### Example 4.9

| Ingredient | Percentage |
|---|---|
| Gum Arabic | 20 |
| Resistant Starch | 5 |
| Psicose | 28 |
| Trehaolse | 13 |
| Isomaltulose | 12 |
| Glycerol | 8 |
| Bees Wax | 3 |
| Carnauba Wax | 1.8 |
| Stannous Fluoride | 0.05 |
| Chlorhexidine | 0.005 |
| Caffeic | 0.005 |
| Lecithin | 0.2 |
| Tea Tree Essential Oil | 0.5 |
| Aloe Vera | 1 |
| Sodium Perborate | 0.3 |
| Vitamin Premix | 0.14 |
| White Oak Bark PDW | 3 |
| Oregon Grape Root | 3 |
| Myrrh | 1 |
| Total | 100 |

1. A semi-solid chewable composition for oral health having a pH of at least 4, comprising
an herbal composition,
an amino acid composition comprising from about 0.5% to about 8% of arginine.
arginine,
a vitamin composition comprising biotin, vitamin D, vitamin C, or a combination thereof,
a pH buffering composition, and
a semi-chewable base,
wherein the semi-solid chewable composition is substantially free of sucrose, fructose and glucose.

2. The semi-solid chewable composition of claim 1, wherein the semi-chewable base comprises a gelling agent selected from a group consisting of pectin, gelatin, agar, carrageenan, starch, gum Arabic, konjac, or a combination thereof, and wherein the composition is configured to dissolve in oral cavity through chewing.

3. The semi-solid chewable composition of claim 2, wherein the semi-chewable base comprises a bonding agent selected from a sugar alcohol, a low glycemic index sugar, or a combination thereof, wherein the sugar alcohols comprise sorbitol, mannitol, erythritol, xylitol, Isomalt, maltitol, lactitol, hydrogenated starch hydrolysates, or a combination thereof, and wherein the low glycemic index sugar comprises psicose, sorbose, tagatose, trehalose, isomaltulose, raffinose or a combination thereof.

4-5. (canceled)

6. The semi-solid chewable composition of claim 1, wherein the semi-chewable base comprises a hydrophobic material and the composition is configured to be non-dissolvable in oral cavity through chewing.

7. The semi-solid chewable composition of claim 6, wherein the hydrophobic material comprises hydrophobic resin, wax, elastomer, or a combination thereof.

8. The semi-solid chewable composition of claim 7, further comprising a hydrophilic polymer selected from gelatin, pectin, carrageenan, guar gum, xanthan gum, gum Arabic, locust bean gum, polyacrylic acid and associated salts, polyethylene glycol, polyethylene oxide, polylactic acid, hydroxyethyl cellulose, carboxymethyl cellulose, or a combination thereof.

9. The semi-solid chewable composition of claim 1, wherein the herbal composition comprises noroginseng, skullcap, isatisis root, dandelion root, corydalis, ginger, Zanthoxylum nitidum (Roxb.), Yan Husuo, Qingfengteng, Sarcandra glabra (Thunb.) Nakai, Imperata cylindrica, Pueraria lobate, Ribes nigrum L., Zanthoxylum nitidum (Roxb.), Rehmannia glutinosa, Mulberry leaf, Guan Gui (cortex cinnamomic), mentha, Centella asiatica (L.) Urban, Pueraria lobate, Lophatherum gracile, Scutellaria baicalensis Georgi, honeysuckle, wild chrysanthemum, chamomile, licorice, ginseng, Ganoderma Lucidum Karst, Ribes nigrum L, Gingko, green tea, pearl powder, powder of mother of pearl, ginger, Fructus armeniaca mume, Porphyromonas gingivalis, Cortex magnoliae officinalis, Cortex phellodendri, Flos caryophylli, Flos lonicerae japonicae, Fructus armeniaca mume, Fructus forsythiae suspensae, Herba cum radice violae yedoensitis, Herba menthae haplocalycis, Pericarpium granati, Radix et rhizoma rhei, Radix gentianae, Ramulus cinnamomi cassia, Rhizoma cimicifugae, or a combination thereof.

10. The semi-solid chewable composition of claim 1, wherein the herbal composition comprises Zanthoxylum nitidum (Roxb.), Centella asiatica, mentha, or a combination thereof.

11. The semi-solid chewable composition of claim 1, wherein the herbal composition comprises Zanthoxylum nitidum (Roxb.), Yanhusuo, Guangui, or a combination thereof.

12. The semi-solid chewable composition of claim 1, wherein the herbal composition comprises Zanthoxylum nitidum (Roxb.), Yanhusuo, Qingfengteng, Rehmannia glutinosa, Mulberry leaf, or a combination thereof.

13. The semi-solid chewable composition of claim 1, wherein the herbal composition comprises Dandelion, Radix Isatidis, Scutellaria baicalensis, or a combination thereof.

14. The semi-solid chewable composition of claim 1, wherein the herbal composition comprises Dandelion Root, Radix Isatis, Corydalis bungeana, Baikal Skullcap, or a combination thereof.

15. The semi-solid chewable composition of claim 1, wherein the herbal composition comprises honeysuckle, scutellaria baicalensis, forsythia, or a combination thereof.

16. The semi-solid chewable composition of claim 1, wherein the herbal composition comprises loptatherum gracile, mint, or a combination thereof.

17. The semi-solid chewable composition of claim 1, wherein the herbal composition comprises chrysanthemum, chamomile, lmperata cylindrinca, or a combination thereof.

18. The semi-solid chewable composition of claim 1, wherein the herbal composition comprises Cortex phellodendri.

19. The semi-solid chewable composition of claim 1, wherein the herbal composition comprises Zanthoxylum nitidum (Roxb.) DC.

20. The semi-solid chewable composition of claim 1, wherein the herbal composition comprises honeysuckle, scutellaria baicalensis, forsythia, or a combination thereof.

21. (canceled)

22. The semi-solid chewable composition of claim 1, further comprising a mineral composition, an anti-caries agent, an anti-calculus agent, a chelating agent, a prebiotics, or a probiotics, wherein the mineral composition comprises a calcium salt, a zinc salt, or a combination thereof.

23-27. (canceled)
